(19) United States
(12) Patent Application Publication
(10) Pub. No.: US 2005/0101605 A1
(43) Pub. Date: May 12, 2005

(54) Oral liquid formulations of methotrexate

(76) Inventors: Salah U. Ahmed, New City, NY (US); Pruthivapathy R. Katikaneni, Boonton, NJ (US); Raj R. Mahajan, Lodi, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/982,770

(22) Filed: Nov. 8, 2004

Related U.S. Application Data
(60) Provisional application No. 60/517,894, filed on Nov. 7, 2003.

(51) Int. Cl. A61K 31/525; A61K 9/00

(52) U.S. Cl. 514/251; 424/400

(57) ABSTRACT

The present invention relates to novel formulations of methotrexate in liquid form that are suitable for oral use.

### BACKGROUND OF THE INVENTION

1. Field of the Invention

The present invention relates to novel formulations of methotrexate in liquid form that are suitable for oral use.

2. Background Art

Methotrexate (4-amino-10-methylfolic acid) is a structural analog of folic acid and inhibits dihydrofolate reductase. Methotrexate and its active metabolites compete for the folate binding site of the enzyme dihydrofolate reductase (DHFR). Folic acid must be reduced to tetrahydrofolic acid by DHFR for DNA synthesis and cellular replication to occur. Competitive inhibition of DHFR leads to blockage of tetrahydrofolate (THF) synthesis, depletion of nucleotide precursors, and inhibition of DNA, RNA and protein synthesis. Methotrexate is cell cycle phase-specific (S phase). Upon prolonged storage of methotrexate, methyl folic acid (also known as methyl folate, MFA) can form. If MFA builds up in the body, anemia can result, through a process called the folate trap.

Methotrexate (formerly amethopterin) is an anti-metabolite used in the treatment of certain arthritic and psoriatic diseases, such as psoriatic arthritis, psoriasis, systemic dermatomyositis, seronegative arthritis, adult rheumatoid arthritis, resistant juvenile rheumatoid arthritis, graft versus host disease, mycosis fungoides, spondyloarthropathy and spondyloarthropathies, including ankylosing spondylitis. Methotrexate is also used in the treatment of neoplasms or cancers such as acute lymphocytic leukemia, breast cancer, bladder cancer, head and neck cancer, non-Hodgkin's lymphoma, osteogenic sarcoma, adult soft tissue sarcoma, choriocarcinoma, lung cancer, and other cancers, or as a supplement for these conditions.

Formulations and drug delivery systems incorporating methotrexate are known in the art. U.S. Pat. No. 4,474,752 discloses an injectable pharmaceutical composition, containing various drugs including methotrexate, which is a liquid at room temperature and a semi-solid gel at body temperature. U.S. Pat. No. 5,472,954 discloses a composition of various drugs, including methotrexate in a solid or liquid co-complex with cyclodextrin. U.S. Pat. No. 5,770,585 discloses methotrexate, or other pharmaceuticals, in a water-in-perfluorochemical liquid dispersion, for treatment of the lung. U.S. Pat. No. 5,925,669 discloses a composition including methotrexate or other antineoplastic agents in a triglyceride, oil-rich in docosahexanoic acid. WO 97/00670 and U.S. Pat. No. 6,083,518 disclose a biologically active agent including methotrexate, a glass-forming substance and a plasticiser. U.S. Pat. No. 6,309,663 discloses a pharmaceutical composition containing at least one hydrophilic surfactant, one hydrophobic surfactant and/or a hydrophilic therapeutic agent, including methotrexate. U.S. Pat. No. 6,383,471 discloses a pharmaceutical composition containing an ionizable hydrophobic therapeutic agent, including methotrexate or other therapeutic agents, a carrier containing a surfactant and an ionizing agent, and a triglyceride.

Methotrexate formulations are commercially available in solid dosage forms as tablets.

### BRIEF SUMMARY OF THE INVENTION

A first aspect of the present invention is directed to an oral liquid pharmaceutical composition for gastrointestinal administration comprising methotrexate and a polyol.

A second aspect of the present invention is directed to method of making an oral liquid methotrexate composition for gastrointestinal administration, the method comprising: (a) combining methotrexate with a suitable solid or liquid polyol, (b) adding a pharmaceutically acceptable carrier, excipient or diluent suitable for oral use, and (c) obtaining an oral liquid methotrexate composition for gastrointestinal administration.

A third aspect of the present invention is directed to a kit comprising (a) a first container means containing a therapeutically effective amount of the oral liquid pharmaceutical composition of the present invention, and (b) a second container means containing a carrier, excipient, diluent or combination thereof.

A fourth aspect of the present invention is directed to a method of administering methotrexate to a patient in need thereof, the method comprising administering to the patient an oral liquid pharmaceutical composition for gastrointestinal administration comprising methotrexate and a polyol.

### BRIEF DESCRIPTION OF THE DRAWINGS/FIGURES

FIG. 1 depicts a process flow chart for the manufacture of an oral liquid formulation of methotrexate.

### DETAILED DESCRIPTION OF THE INVENTION

There exists a need to provide an oral liquid formulation of methotrexate for the ease in administration of the agent, particularly in children, in patients 55 years of age or older, or other persons that can have difficulty or an inability to swallowing (dysphagia) the methotrexate tablets currently available. There also exists a need for such an oral liquid form of methotrexate for veterinary usage.

The present invention provides an oral liquid pharmaceutical composition for gastrointestinal administration comprising methotrexate, or a pharmaceutically acceptable salt or ester thereof, and a polyol.

Methotrexate is a structural analog of folic acid and is represented by Formula I:

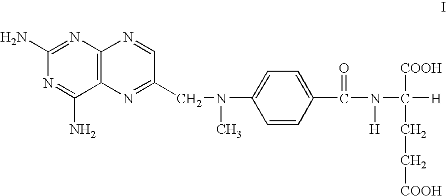

Methotrexate is also known by its chemical names N-[4-[[(2,4-diamino-6-pteridinyl)methyl]methylamino]benzoyl]-L-glutamic acid and 4-amino-10-methylfolic acid. As used herein, methotrexate also refers to pharmaceutically acceptable salts or esters of methotrexate. Pharmaceutically acceptable salts can be selected from, but are not limited to, alkali metal salts such as sodium or potassium, alkaline earth salts or an ammonium salt (all of which are herein referred to as a pharmaceutically acceptable salts). In some embodiments, methotrexate refers to methotrexate disodium. Methotrexate also refers to acetylated forms, benzhydryl-sulfinylacetic acid forms, sulfone forms, hydroxylated forms, polymorphic forms, analogs, derivatives, cogeners, prodrugs, metabolic acids and compounds made by mixtures thereof. The methotrexate of the present invention also includes individual enantiomers or racemic mixtures of methotrexate.

The oral liquid pharmaceutical compositions of the present invention are administered orally as a liquid. In the present invention, liquid refers to a composition in a fluid state, which has no independent shape but has a definite volume and does not expand indefinitely. Examples of liquid dosage forms include, but are not limited to, solutions, suspensions, emulsions, elixirs and/or aerosols.

In the present invention, oral refers to delivery of the pharmaceutical composition via, or involving, the mouth.

The oral liquid pharmaceutical composition of the present invention is for administration to the gastrointestinal tract of the subject being treated. Gastrointestinal administration includes administration to the stomach and intestines. In some embodiments, the oral liquid pharmaceutical composition of the present invention is for administration to the stomach. In some embodiments, the oral liquid pharmaceutical composition of the present invention is for administration to the intestines. For example, administration of the oral liquid pharmaceutical composition of the present invention can occur by the subject orally swallowing the composition of the present invention, thereby administering the composition of the present invention to the stomach and the intestines.

Pharmaceutically acceptable salts refers to salts of methotrexate which are, within the scope of sound medical judgement, suitable for contact with the tissues of human beings and animals without excessive toxicity, irritation, allergic response, or other problem complications commensurate with a reasonable benefit/risk ratio.

The composition of the invention can include a polyol, which is a nonionic hydrophilic surfactant. The polyol can be selected from, but is not limited to, glycerin, polyethylene glycol, sorbitol, propylene glycol, pentaerythritol, sodium-saccharin and combinations thereof. In some embodiments of the invention, the polyol can be propylene glycol. In some embodiments, the polyol can be glycerin.

In some embodiments, the polyol can be a mixture of propylene glycol and glycerin. Various ratios of propylene glycol and glycerin can be used in the present invention. In some embodiments, the polyol can be about a 1:1 mixture of propylene glycol and glycerin, respectively. In some embodiments, the polyol can be about a 2:1 mixture of propylene glycol and glycerin, respectively. In some embodiments, the polyol can be about a 5:1 mixture of propylene glycol and glycerin, respectively. In some embodiments, the polyol can be about a 10:1 mixture of propylene glycol and glycerin, respectively. In some embodiments, the polyol can be about a 1:2 mixture of propylene glycol and glycerin, respectively. In some embodiments, the polyol can be about a 1:5 mixture of propylene glycol and glycerin, respectively. In some embodiments, the polyol can be about a 1:10 mixture of propylene glycol and glycerin, respectively.

The pH of the oral liquid pharmaceutical composition of the present invention can vary. In some embodiments, the pH of the present invention can be between about 6.0 to about 8.0. In some embodiments, the pH of the present invention can be between about 6.5 and 7.5. In some embodiments, the pH of the present invention can be between about 6.8 to about 7.2. In some embodiments, the pH of the present invention can be about 7.0.

The composition of the invention also can contain preservatives such as, but not limited to, methylparaben (also known as 4-hydroxybenzoic acid methyl ester, methyl p-hydroxybenzoate, Nipagin M, Tegosept M, Methyl Parasept or Methyl Chemosept), ethylparaben (also known as 4-hydroxybenzoic acid ethyl ester, ethyl p-hydroxybenzoate or Ethyl Parasept), propylparaben (also known as 4-hydroxybenzoic acid propyl ester, propyl p-hydroxybenzoate, Nipasol or Propyl Chemosept), butylparaben (also known as 4-hydroxybenzoic acid propyl ester, propyl p-hydroxybenzoate or Butyl Chemosept), or combinations thereof. In some embodiments, the composition can contain methylparaben and/or propylparaben.

Pharmaceutical compositions of methotrexate can accumulate methyl folic acid during storage. In some embodiments of the present invention, the accumulated methyl folic acid concentration in the oral liquid composition is less than about 2% when the composition is stored for about three months at about 40° C. In some embodiments of the present invention, the accumulated methyl folic acid concentration in the oral liquid composition is less than about 2% when the composition is stored for about 2 years at about 25° C. In some embodiments, the accumulated methyl folic acid concentration in the oral liquid composition is less than about 1% when the composition is stored for about three months at about 40° C., or for about 2 years at about 25° C.

In the present invention, the concentration of methotrexate can vary. In some embodiments, the oral liquid pharmaceutical composition contains from about 1 mg/mL to about 10 mg/mL of methotrexate. In some embodiments, the oral liquid pharmaceutical composition contains about 2.5 mg/mL to about 10 mg/mL or about 2.5 mg/mL to about 5 mg/mL. In some embodiments, the composition of the invention contains about 2.5 mg/mL of methotrexate. In some embodiments, the composition of the invention contains about 5.0 mg/mL of methotrexate.

In some embodiments, the composition of the invention contains from about 1 mg/mL to about 10 mg/mL of methotrexate disodium. In some embodiments, the composition of the invention contains about 2.5 mg/mL to about 10 mg/mL of methotrexate disodium. In some embodiments, the composition of the invention contains about 2.5 mg/mL to about 5 mg/mL of methotrexate disodium. In some embodiments, the composition of the invention contains about 2.5 mg/mL of methotrexate. In some embodiments, the composition of the invention contains about 5 mg/mL of methotrexate disodium.

In the present invention, the concentration of the polyol can vary. In some embodiments, the oral liquid pharmaceutical composition contains from about 10 mg/mL to about 990 mg/mL polyol. In some embodiments, the composition contains from about 25 mg/mL to about 950 mg/mL polyol. In some embodiments, the composition contains from about 500 mg/mL to about 950 mg/mL polyol. In some embodiments, the composition contains about 940 mg/mL polyol. In some embodiments, the polyol is propylene glycol. In some embodiments, the composition contains from about 10 mg/mL to about 900 mg/mL propylene glycol. In some embodiments, the composition contains from about 25 mg/mL to about 700 mg/mL propylene glycol. In some embodiments, the composition contains from about 100 mg/mL to about 500 mg/mL propylene glycol. In some embodiments, the composition contains about 100 mg/mL propylene glycol. In some embodiments, the composition contains about 500 mg/mL propylene glycol. In some embodiments, the polyol is glycerin. In some embodiments, the composition contains from about 10 mg/mL to about 900 mg/mL glycerin. In some embodiments, the composition contains from about 25 mg/mL to about 700 mg/mL glycerin. In some embodiments, the composition contains from about 100 mg/mL to about 550 mg/mL glycerin. In some embodiments, the composition contains about 540 mg/mL glycerin.

In some embodiments, the oral liquid composition of the present invention further contains a pharmaceutically acceptable carrier, diluent, excipient and/or combination thereof. As used herein, the term “excipient” or “carrier” refers to additives used for preparing a convenient or pharmaceutically acceptable dosage form, or for facilitating the processing of the active compounds into preparations that can be administered to animals, as described herein. For compositions of the present invention suitable for administration to a human, the terms “excipient” and “carrier” include those excipients and carriers described in the Handbook of Pharmaceutical Excipients, American Pharmaceutical Association, 2^{nd} Ed. (1994), which is herein incorporated in its entirety. The terms “excipient” and “carrier” are meant to include, but are not limited to, diluents, flavoring agents, sweeteners, preservatives, solvents, buffers, dyes, extenders, edetate disodium, methylparaben, ethylparaben, propylparaben, butylparaben, citric acid, sodium citrate, water, ethanol and combinations thereof and the like. Liquid excipients include, but are not limited to, various oils, including those of petroleum, animal, vegetable or synthetic origin, such as, peanut oil, soybean oil, mineral oil, sesame oil, hydrogenated vegetable oil, cottonseed oil, groundnut oils, corn oil, germ oil, olive oil, castor oil, and so forth. In some embodiments, the pharmaceutically acceptable carrier can be, but is not limited to, coloring agents, ethanol, EDTA, citrate buffer, flavoring, water and combinations thereof. In some embodiments, the oral liquid pharmaceutical composition comprises EDTA, citrate buffer, flavoring and water.

The oral liquid pharmaceutical composition of the invention can be combined with various sweeteners or flavoring agents such as, but not limited to, orange or lemon flavors. Sweeteners include, but are not limited to, sucrose, fructose, sodium saccharin, xylitol, sorbitol, mannitol, aspartame (also known as NUTRA-SWEET®), sucralose (also known as SPLENDA®), sodium cyclamate and combinations thereof. In some embodiments, the sweetener is aspartame, sucralose, sodium cyclamate, sodium saccharin or combinations thereof. In some embodiments, diluents such as water, glycerin and combinations thereof are added to the sweeteners.

Various diluents can be used in the present invention. A diluent can be considered as any inert substance, or mixture of substances, added to increase the volume of the pharmaceutical formulation in order to make the oral liquid pharmaceutical composition of the present invention a practical size for administration. In some embodiments, the diluent is water. Various amounts of water can be added to the present invention. In some embodiments, the water added is from about 10 mg/mL to about 150 mg/mL. In some embodiments, the composition of the present invention further comprises water in an amount equal to or less than about 100 mg/mL.

In some embodiments, coloring agents such as dye stuffs, natural coloring agents or pigments can be combined with the oral liquid pharmaceutical composition of the present invention. In some embodiments, diluents such as water, glycerin and combinations thereof can be combined with the coloring agents.

Suitable excipients also include, but are not limited to, fillers such as saccharides, for example, lactose, fructose, sucrose, inositol, mannitol or sorbitol, xylitol, trehalose, cellulose preparations and/or calcium phosphates.

In some embodiments, the oral liquid pharmaceutical composition comprising methotrexate is in the form of an aerosol. A methotrexate solution for aerosol administration is formulated to achieve localized delivery to the lungs. This is accomplished by preparing an aqueous aerosol, liposomal preparation or solid particles containing methotrexate and/or other pharmaceutically active agents. Ordinarily, an aqueous aerosol is made by formulating an aqueous solution or suspension of methotrexate together with conventional pharmaceutically acceptable carriers and stabilizers. The carriers and stabilizers vary depending upon the requirements for the delivery of methotrexate, but typically include: nonionic surfactants; proteins such as serum albumin, sorbitan esters, oleic acid, lecithin; amino acids such as glycine; and buffers, salts, sugars or sugar alcohols. The formulations can also include mucolytic agents as well as bronchodilating agents. In some embodiments, an aerosol formulation of the present invention does not include sweeteners or flavorings. The formulations can be sterile. Aerosols generally are prepared from isotonic solutions. The composition can optionally include normal lung surfactants.

The compositions of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, pharmaceutically acceptable and metabolizable lipid capable of forming liposomes can be used. The present compositions in liposome form can contain, in addition to the compounds of the present invention, stabilizers, preservatives, excipients, and the like. The preferred lipids are the phospholipids and the phosphatidyl cholines (lecithins), both natural and synthetic. Methods to form liposomes are known in the art (see, for example, Prescott, ed., Meth. Cell Biol. 14:33 (1976)).

The oral liquid pharmaceutical composition of the present invention can also include one or more additional therapeutic agents such as, but not limited to, hydrophilic drugs, hydrophobic drugs, hydrophilic macromolecules, cytokines, peptidomimetics, peptides, proteins, toxoids, sera, antibodies, vaccines, nucleosides, nucleotides, nucleoside analogs, genetic materials and/or combinations thereof. Additional examples of therapeutic agents that can be used in the pharmaceutical compositions of the present invention include, but are not limited to, other antineoplastic agents, analgesics and anti-inflammatory agents, anti-anginal agents, antihelmintics, anti-arrythmic agents, anti-arthritic agents, anti-asthma agents, anti-bacterial agents, anti-viral agents, antibiotics, anti-coagulants, anti-depressants, antidiabetic agents, anti-epileptic agents, anti-emetics, anti-fungal agents, anti-gout agents, anti-hypertensive agents, anti-malarial agents, antimigraine agents, anti-muscarinic agents, anti-parkinson's agents, anti-protozoal agents, anti-thyroid agents, thyroid therapeutic agents, anti-tussives, anxiolytic agents, hypnotic agents, neuroleptic agents, β-blockers, cardiac inotropic agents, corticosteroids, diuretics, gastrointestinal agents, histamine H-receptors antagonists, immunosuppressants, keratolytics, lipid regulating agents, muscle relaxants, nutritional agents, cytokines, peptidomimetics, peptides, proteins, toxoids, sera, sedatives, sex hormones, sex hormone antagonists or agonists, stimulants antibodies, vaccines, nucleosides, nucleoside analogs and genetic materials. Amphiphilic therapeutic agents and nutritional agents can also be included.

The additional therapeutic agent can be solubilized or suspended in a preconcentrate (before dilutions with a diluent), added to the preconcentrate prior to dilution, added to the diluted preconcentrate, or added to a diluent prior to mixing with the preconcentrate. The additional therapeutic agent can also be co-administered as part of an independent dosage form, for therapeutic effect. Optionally, the additional therapeutic agent(s) can be present in a first, solubilized amount, and a second, non-solubilized (suspended) amount. Such additional therapeutic agent(s) can be any agent(s) having therapeutic or other value when administered to an animal, particularly to a mammal, such as drugs, nutrients, and diagnostic agents.

The pharmaceutical compositions of the present invention can be administered to any mammal that can experience the beneficial effects of the compounds of the invention. Such mammals include humans and non-humans, such as pets and farm animals. The dosage administered will be dependent upon the age, health, and weight of the recipient, kind of concurrent treatment, if any, frequency of treatment, and the nature of the effect desired. Pharmaceutical formulations useful in the present invention can contain a quantity of a compound(s) according to this invention in an amount effective to treat the condition, disorder or disease of the subject being treated.

The invention is also directed to a kit comprising the oral liquid pharmaceutical composition of the present invention. In some embodiments, the kit comprises (a) a first container means containing a therapeutically effective amount of the oral liquid pharmaceutical composition of the present invention and (b) a second container means containing a pharmaceutically acceptable carrier, excipient, diluent or combination thereof. Optionally, the kit can have additional container mean(s) comprising a therapeutically effective amount of additional agents.

In some embodiments, the kit comprises a container for the separate compositions such as a divided bottle or a divided foil packet, however, the separate compositions can also be contained within a single, undivided container. Typically, the kit contains directions for administration of the separate components. The kit form is particularly advantageous when the separate components are preferably administered at different dosage intervals, or when titration of the individual components of the combination is desired by the prescribing physician.

In some embodiments, the kit of the present invention can further comprise an additional container means comprising a therapeutically effective amount of an agent selected from the group consisting of hydrophilic drugs, hydrophobic drugs, hydrophilic macromolecules, cytokines, peptidomimetics, peptides, proteins, toxoids, sera, antibodies, vaccines, nucleosides, nucleotides, nucleoside and/or nucleotide analogs, genetic materials and combinations thereof.

Methods of Preparation

The present invention also provides for a method of making an oral liquid methotrexate composition for gastrointestinal administration, the method comprising (a) combining methotrexate with a suitable solid or liquid polyol, (b) adding a pharmaceutically acceptable carrier, excipient or diluent suitable for oral use and (c) obtaining an oral liquid methotrexate composition for gastrointestinal administration.

Methods of preparing various pharmaceutical compositions with a certain amount of active ingredients are known, or will be apparent in light of this disclosure, to those skilled in the art. Methods of preparing the pharmaceutical compositions can incorporate other suitable pharmaceutical excipients and their formulations as described in Remington's Pharmaceutical Sciences, Martin, E. W., ed., Mack Publishing Company, 19^{th} ed. (1995).

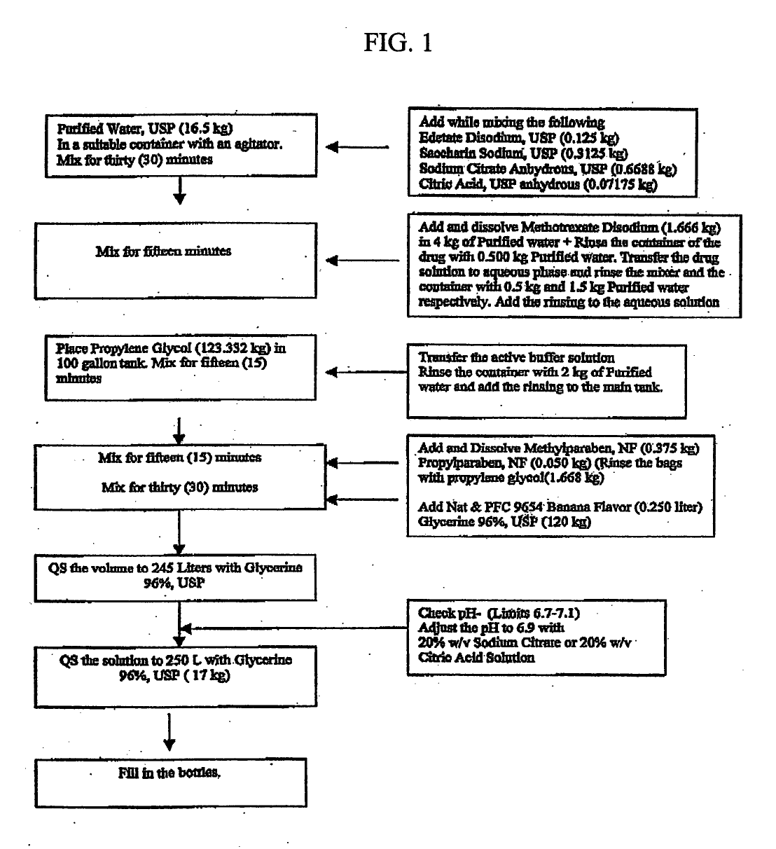

Methods of preparing the pharmaceutical preparations of the present invention are manufactured in a manner that is known, including conventional mixing, dissolving, or lyophilizing processes. Thus, oral liquid pharmaceutical preparations can be obtained by combining the active compounds with other solid or liquid excipients, optionally mixing the resulting mixture and processing the mixture of solution, after adding suitable auxiliaries, if desired or necessary. One exemplary method of preparing the pharmaceutical preparations of the present invention is depicted in FIG. 1, wherein methotrexate is mixed with propylene glycol and glycerin, and other ingredients as described in Tables 1-3.

Methods of Treatment

The invention is directed to a method of administering methotrexate to a patient in need thereof, the method comprising administering to the patient an oral liquid pharmaceutical composition for gastrointestinal administration comprising methotrexate. In some embodiments, the present invention provides methods of treating a patient in need thereof, comprising administering to the patient the oral liquid composition of methotrexate for the treatment of psoriasis, psoriatic arthritis, systemic dermatomyositis, seronegative arthritis, adult rheumatoid arthritis, resistant juvenile rheumatoid arthritis, graft versus host disease, mycosis fungoides, spondyloarthropathy, spondyloarthropathies, including ankylosing spondylitis, in the treatment of neoplasms or cancers such as acute lymphocytic leukemia, breast cancer, bladder cancer, head and neck cancer, non-Hodgkin's lymphoma, osteogenic sarcoma, adult soft tissue sarcoma, choriocarcinoma, lung cancer, or other cancers or as a supplement for these conditions.

The terms “treat” and “treatment” refer to both therapeutic treatment and prophylactic or preventative measures, wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms; diminishment of extent of condition, disorder or disease; stabilization (i.e., not worsening) of the state of condition, disorder or disease; delay in onset or slowing of condition, disorder or disease progression; amelioration of the condition, disorder or disease state, remission (whether partial or total), whether detectable or undetectable; or enhancement or improvement of condition, disorder or disease. Treatment includes eliciting a clinically significant response, without excessive levels of side effects. Treatment also includes prolonging survival as compared to expected survival if not receiving treatment.

Various patients can find utility in the present invention. In some embodiments, the patient is in need of an oral liquid dosage form of methotrexate. In some embodiments, the patient is a child. In some embodiments, the patient is about 55 years of age or older. In some embodiments, the patient has dysphagia.

One of ordinary skill in the art will appreciate that a method of administering pharmaceutically effective amounts of the methotrexate to a patient in need thereof can be determined empirically, or by standards currently recognized in the medical arts. The agents can be administered to a patient as pharmaceutical compositions in combination with one or more pharmaceutically acceptable carriers, excipients, diluents or combinations thereof. It will be understood that, when administered to, for example, a human patient, the total daily usage of the agents of the compositions of the present invention will be decided within the scope of sound medical judgement by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of the agents at levels lower than those required to achieve the desired therapeutic effect and to gradually increase the dosages until the desired effect is achieved.

Dosaging

Dosaging can also be administered in a patient-specific manner to provide a predetermined concentration of the agents in the blood, as determined by techniques accepted and routine in the art.

For example, satisfactory results for treating neoplasms are obtained by oral administration of the compounds at dosages on the order of from about 1 mg/m² to about 15 g/m², more preferably from about 2.0 to about 500 mg/m², and most preferably from about 2.5 to about 60 mg/m². Suitable dosages for patients vary from about once every week to about once every 4 weeks, or every day for 5 consecutive days. Multiple courses can be given, depending upon parameters well known in the art and to the physician treating the patient. Alternatively, for psoriasis or for rheumatoid arthritis, for example, about 2.5 mg to about 5 mg can be given every 12 hours for 3 doses per week or from about 7.5 to about 25 mg once per week, or other dosing schedules known in the art.

### EXAMPLES

### Example 1

Initial formulations of methotrexate investigated the effect of pH on the stability of an oral liquid pharmaceutical composition of methotrexate by varying the pH of various methotrexate compositions.

Glycerin and propylene glycol were used as polyols because of their low peroxide content. The amount of propylene glycol and glycerin combined was 25% in each of the formulations, and the sweetener concentration was 50% (Table 1). Sucrose (extra fine granulated) was used as the sweetener. The formulations were adjusted to a pH range of 6 to 7.5 and the water content was about 25%. The use of a solid sugar requires more water to solubilize all of the water-soluble components. These formulations, when stored at accelerated conditions, resulted in amounts of methylfolic acid greater than 2%.

TABLE 1

| | mg/dose | | | | | |
|---|---|---|---|---|---|---|
| Composition | Batch 1 | | Batch 2 | | Batch 3 | |
| Methotrexate | 2.5 | | 2.5 | | 2.5 | |
| Disodium | | | | | | |
| Propylene Glycol, | 150 | | 150 | | 150 | |
| Glycerin, USP | 100 | | 100 | | 100 | |
| Sucrose, NE | 500 | | 500 | | 500 | |
| (Sugar Extra Fine | | | | | | |
| Granulated) | | | | | | |
| Edetate Disodium, | 0.5 | | 0.5 | | 0.5 | |
| USP | | | | | | |
| Methylparaben, NF | 1.5 | | 1.5 | | 1.5 | |
| Propylparaben, NF | 0.2 | | 0.2 | | 0.2 | |
| Citric | 0.50 | | 0.25 | | — | |
| Acid, Anhydrous, USP | | | | | | |
| Sodium Citrate, | 4.0 | | 2.0 | | 8.0 | |
| Anhydrous, USP | | | | | | |
| Flavor | 0.01 | | Lemon-0.01 | | Orange | |
| Purified Water, USP | Qs to 1 mL | | Qs to 1 mL | | Qs to 1 mL | |
| | (e.g., 458 mg) | | (e.g., 440 mg) | | (e.g., 454 mg) | |
| TOTAL | 1 mL | | 1 mL | | 1 mL | |
| PH | 6.2 | | 6.7 | | 7.5 | |
| | Assay % | | Assay % | | Assay % | |
| | MTX | MFA | MTX | MFA | MTX | MFA |
| Initial | 98.44 | — | 103.7 | 0.47 | | |
| 2 week at 40° C. | — | — | | 2.82 | 100 | — |
| 3 month at RT | — | 0.61 | | | | |
| 3 month at 40° C. | 95 | 2.62 | | | | 2.49 |
| 1 week at 60° C. | — | — | | 1.92 | 93.3 | — |
| MTX designates methotrexate. Batch 1 showed a MFA concentration of 2.6% after 3 months of storage at 40° C. Batch 2 showed a MFA concentration of 2.82% after 2 weeks of storage at 40° C. | | | | | | |
| # Batch 3 showed a MFA concentration of 2.49% and total impurities were 2.82% after 3 months of storage at 40° C. | | | | | | |

### Example 2

In subsequent formulations of methotrexate, the amounts of glycerin and propylene glycol were varied and the amount of water was reduced to 10% (Tables 2 and 3). After 1 month at an accelerated temperature of 60° C., the MFA concentration was 3.12% in the methotrexate formulation containing glycerin. However, after storage for 1 month at an accelerated temperature of 60° C., the MFA concentration was 0.75% in the methotrexate formulation containing propylene glycol. Methotrexate formulations containing either glycerin or propylene glycol contained low concentrations of MFA (0.63% and 0.34%, respectively) after storage for 1 month at 40° C. (Table 2).

TABLE 2

| | mg/dose | | | |
|---|---|---|---|---|
| Composition | Batch 4 (2.5 mg/mL) | | Batch 5 (2.5 mg/mL) | |
| Methotrexate Disodium | 2.5 | | 2.5 | |
| Propylene Glycol, USP | Qs to 1 mL | | — | |
| | (e.g., 840 mg) | | | |
| Glycerin, USP | — | | Qs to 1 mL | |
| | | | (e.g., 570 mg) | |
| Edetate Disodium, USP | 0.5 | | 0.5 | |
| Methylparaben, NF | 1.5 | | 1.5 | |
| Propylparaben, NF | 0.2 | | 0.2 | |
| Citric Acid, Anhydrous, | 0.5 | | 0.5 | |
| USP | | | | |
| Sodium Citrate, | 4.0 | | 4.0 | |
| Anhydrous, USP | | | | |
| Purified Water, USP | 100 | | 100 | |
| TOTAL | 1 mL | | 1 mL | |
| PH | 7.0 | | 6.5 | |
| | Assay % | | Assay % | |
| | MTX | MFA | MTX | MFA |
| Initial | 100 | 0.31 | 100.0 | 0.37 |
| 15 days at 60° C. | 96.6 | 0.60 | 97.4 | 0.85 |
| 1 Month at 40° C. | 95.7 | 0.34 | 103.6 | 0.63 |
| 1 Month at 60° C. | 94.5 | 0.75 | 88.7 | 3.12 |

Formulations containing a combination of propylene glycol and glycerin were investigated. In a methotrexate formulation containing a 1:1 mixture of propylene glycol and glycerin, the concentration of MFA was 0.29%-0.35% after storage at 40° C. for 1 month (Table 3). This concentration of MFA is similar to the concentration of MFA in formulations containing propylene glycol alone after similar storage conditions, and is less than the MFA concentration in methotrexate formulations containing glycerin alone. The 1:1 methotrexate formulation is more palatable compared to the formulation containing propylene glycol alone. The effect of the 1:1 mixture of propylene glycol and glycerin was similar in formulations containing different concentrations of methotrexate (Table 3). The potency for batch 6 was 2.5 mg/mL, while the potency for batch 7 was 5 mg/mL.

It was observed that at above pH 6.5, the only degradation product was N¹⁰-methylpteroglutamic acid, while below this pH, the route of degradation is more complex and several compounds are formed.

TABLE 3

| | 2.5 mg/mL | | | 5 mg/mL | | |
|---|---|---|---|---|---|---|
| Composition | Batch 6 | | | Batch 7 (mg/dose) | | |
| Methotrexate | 2.5 | | | 5 | | |
| Disodium | | | | | | |
| Propylene | 500 | | | 500 | | |
| Glycol, USP | | | | | | |
| Glycerin, USP | Qs to 1 mL | | | Qs to 1 mL | | |
| | (e.g., 540 mg) | | | (e.g., 540 mg) | | |
| Saccharin | 1.25 | | | 1.25 | | |
| Sodium, USP | | | | | | |
| Edetate | 0.5 | | | 0.5 | | |
| Disodium, USP | | | | | | |
| Methylparaben, NF | 1.5 | | | 1.5 | | |
| Propylparaben, NF | 0.2 | | | 0.2 | | |
| Citric Acid, | 0.40 | | | 0.287 | | |
| Anhydrous, USP | | | | | | |
| Sodium Citrate, | 3.50 | | | 2.667 | | |
| Anhydrous, USP | | | | | | |
| PFC 9654 | 0.001 mL | | | 0.001 mL | | |
| Banana Flavor | | | | | | |
| Purified Water, USP | 100 | | | 100 | | |
| TOTAL | 1 mL | | | 1 mL | | |
| PH | 7.0 | | | 7.0 | | |
| | Assay % | | | Assay % | | |
| | | | Total | | | Total |
| | MTX | MFA | Impurities | MTX | MFA | Impurities |
| Initial | 100.3 | 0.19 | 0.38 | 97.5 | 0.26 | 0.31 |
| 1 Month at 40° C. | 98.8 | 0.29 | 1.38 | 97.5 | 0.35 | 1.20 |
| 2 Month at 40° C. | 97.1 | 0.40 | 2.56 | 95.7 | 0.49 | 2.38 |
| 3 Month at 40° C. | 96.2 | 0.55 | 3.60 | 92.7 | 0.61 | 3.53 |

The reduction in the methotrexate concentration was only 4.8% (from 97.5% to 92.7%); the concentration of MFA was only 0.61% after 3 months storage at an accelerated temperature of 40° C. The total impurities were 3.53%.

These examples illustrate possible formulations of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. An oral liquid pharmaceutical composition for gastrointestinal administration comprising methotrexate of Formula I: 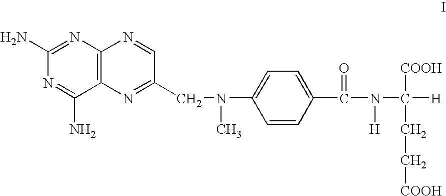
or a pharmaceutically acceptable salt or ester thereof, and a polyol.

2. The composition of claim 1, wherein said methotrexate is methotrexate disodium.

3. The composition of claim 1 wherein said polyol is selected from the group consisting of glycerin, polyethylene glycol, sorbitol, propylene glycol, pentaerythritol, sodium saccharin and combinations thereof.

4. The composition of claim 1, wherein said polyol is propylene glycol.

5. The composition of claim 1, wherein said polyol is glycerin.

6. The composition of claim 1, wherein said polyol is about a 1:1 mixture of propylene glycol and glycerin.

7. The composition of claim 3, further comprising methylparaben and/or propylparaben.

8. The composition of claim 1, wherein the accumulated methyl folic acid concentration in said oral liquid pharmaceutical composition is less than about 2% when said composition is stored for about three months at about 40° C.

9. The composition of claim 1, wherein the accumulated methyl folic acid concentration in said oral liquid pharmaceutical composition is less than about 2% when said composition is stored for about 2 years at 25° C.

10. The composition of claim 1, wherein the methotrexate concentration is about 1 mg/mL to about 10 mg/mL.

11. The composition of claim 1, wherein the methotrexate concentration is about 2.5 mg/mL.

12. The composition of claim 1, wherein the methotrexate concentration is about 5 mg/mL.

13. The composition of claim 4, wherein the propylene glycol concentration is about 100 mg/mL.

14. The composition of claim 4, wherein the propylene glycol concentration is about 500 mg/mL.

15. The composition of claim 1 comprising about 1 mg/mL to about 10 mg/mL methotrexate disodium and about 25 mg/mL to about 700 mg/mL propylene glycol.

16. The composition of claim 15 further comprising EDTA, citrate buffer, flavoring and water.

17. The composition of claim 16 further comprising a sweetener.

18. The composition of claim 17, wherein said sweetener is selected from the group consisting of sucrose, fructose, sodium saccharin, sorbitol, mannitol, aspartame, sucralose, sodium cyclamate and combinations thereof.

19. The composition of claim 18, wherein said sweetener is aspartame, sucralose, sodium cyclamate, sodium saccharin or combinations thereof.

20. The composition of claim 16 comprising about 2.5 mg/mL to about 10 mg/mL methotrexate disodium and about 25 mg/mL to about 700 mg/mL propylene glycol.

21. The composition of claim 16, further comprising methylparaben and/or propylparaben.

22. The composition of claim 21, further comprising water in an amount equal to or less than about 100 mg/mL.

23. The composition of claim 1, further comprising a therapeutic agent selected from the group consisting of hydrophilic drugs, hydrophobic drugs, hydrophilic macromolecules, cytokines, peptidomimetics, peptides, proteins, toxoids, sera, antibodies, vaccines, nucleosides, nucleotides, nucleoside analogs, genetic materials and combinations thereof.

24. The composition of claim 1 which is in a dosage form selected from the group consisting of a solution, suspension, emulsion, elixir and aerosol.

25. The composition of claim 1, further comprising a pharmaceutically acceptable carrier selected from the group consisting of edetate disodium, methylparaben, ethylparaben, propylparaben, butylparaben, citric acid, sodium citrate, sweeteners, flavoring agents, coloring agents, water, ethanol and combinations thereof.

26. A method of making an oral liquid methotrexate composition for gastrointestinal administration, said method comprising:
(a) combining methotrexate with a suitable solid or liquid polyol;
(b) adding a pharmaceutically acceptable carrier, excipient, or diluent suitable for oral use; and
(c) obtaining an oral liquid methotrexate composition for gastrointestinal administration.

27. A kit comprising:
(a) a first container means containing a therapeutically effective amount of the oral liquid pharmaceutical composition of claim 1; and
(b) a second container means containing a pharmaceutically acceptable amount of a carrier, excipient, diluent or combination thereof.

28. The kit of claim 27 further comprising an additional container means comprising a therapeutically effective amount of an agent selected from the group consisting of hydrophilic drugs, hydrophobic drugs, hydrophilic macromolecules, cytokines, peptidomimetics, peptides, proteins, toxoids, sera, antibodies, vaccines, nucleosides, nucleotides, nucleoside and/or nucleotide analogs, genetic materials and combinations thereof.

29. A method of administering methotrexate to a patient in need thereof, said method comprising administering to said patient an oral liquid pharmaceutical composition for gastrointestinal administration comprising methotrexate and a polyol.

30. The method of claim 29, wherein said patient has a condition selected from the group consisting of: psoriasis, psoriatic arthritis, systemic dermatomyositis, seronegative arthritis, adult rheumatoid arthritis, resistant juvenile rheumatoid arthritis, graft versus host disease, mycosis fungoides, spondyloarthropathy, spondyloarthropathies, ankylosing spondylitis, neoplasms, acute lymphocytic leukemia, breast cancer, bladder cancer, head cancer, neck cancer, non-Hodgkin's lymphoma, osteogenic sarcoma, adult soft tissue sarcoma, choriocarcinoma and lung cancer.

31. The method of claim 29, wherein said patient is in need of an oral liquid dosage form.

32. The method of claim 31, wherein said patient is a child.

33. The method of claim 31, wherein said patient is about 55 years of age or older.

34. The method of claim 31, wherein said patient has dysphagia.
